(19) United States
(12) Patent Application Publication
(10) Pub. No.: US 2022/0061644 A1
(43) Pub. Date: Mar. 3, 2022

(54) HOLOGRAPHIC ENDOSCOPE

(71) Applicant: Nokia Technologies Oy, Espoo (FI)

(72) Inventors: Nicolas Fontaine, Keyport, NJ (US); David Neilson, Old Bridge, NJ (US); Haoshuo Chen, Aberdeen, NJ (US); Roland Ryf, Aberdeen, NJ (US)

(73) Assignee: Nokia Technologies Oy, Espoo (FI)

(21) Appl. No.: 17/216,184

(22) Filed: Mar. 29, 2021

Related U.S. Application Data
(60) Provisional application No. 63/070,978, filed on Aug. 27, 2020.

(51) Int. Cl. A61B 1/07; A61B 1/06

(52) U.S. Cl. CPC A61B 1/07; A61B 1/0646; A61B 1/063; A61B 1/0661

(57) ABSTRACT

An optical imaging system capable of performing holographic imaging through a multimode optical fiber. Images of an object acquired by the system using different object-illumination conditions can advantageously be used to obtain a holographic image with reduced speckle contrast therein. Additionally, a beat-frequency map of the object acquired by the system using optical-reflectometry measurements therein can be used to augment the depth information of the holographic image for more-detailed three-dimensional rendering of the object for the user. Digital back-propagation techniques may be applied to reduce blurring in the holographic image and in the depth information caused, e.g., by modal dispersion and mode mixing in the multimode optical fiber. Some embodiments may also provide the capability for polarization-sensitive holographic imaging in different spectral regions of light. An example embodiment of the disclosed optical imaging system may be used as a holographic endoscope for medical or industrial applications.

### CROSS-REFERENCE TO RELATED APPLICATIONS

This application claims the benefit of U.S. Provisional Patent Application No. 63/070,978, filed on 27 Aug. 2020, and entitled “HOLOGRAPHIC ENDOSCOPE,” which is incorporated herein by reference in its entirety.

### BACKGROUND

### Field

Various example embodiments relate to optical imaging and, more specifically but not exclusively, to optical endoscopes.

### Description of the Related Art

This section introduces aspects that may help facilitate a better understanding of the disclosure. Accordingly, the statements of this section are to be read in this light and are not to be understood as admissions about what is in the prior art or what is not in the prior art.

In the field of medicine, endoscopy involves the insertion of a long, thin tube directly into the bodily cavity to observe an internal organ or tissue in detail. Endoscopes can also be used for other than medical purposes, e.g., for inspecting machines or tightly confined spaces in industrial settings.

Holography is a technique that enables a light field to be recorded and later reconstructed, e.g., when the original light field is no longer present. A hologram is a physical recording, analog or digital, of an interference pattern of two coherent light waves that can be used to reproduce the original three-dimensional light field, resulting in an image retaining the depth, parallax, and some other characteristics of the recorded scene.

### SUMMARY OF SOME SPECIFIC EMBODIMENTS

Disclosed herein are various embodiments of an optical imaging system capable of performing holographic imaging through a multimode optical fiber. Images of an object acquired by the system using different object-illumination conditions, e.g., differing in one or more of phase, angle, polarization, modal composition, and wavelength of the illumination light, can advantageously be used to obtain a holographic image with reduced speckle contrast therein. Some embodiments of the imaging system may be operated to produce images or aid in the generation of certain images by scanning the surface of the object with a focused illumination beam, with the focusing and scanning being performed by selecting and changing the modal composition of the illumination light in a multimode or multi-core optical fiber. Additionally, a beat-frequency map of the object acquired by the system using optical reflectometry measurements therein can be used to augment the depth information of the holographic image for more-detailed three-dimensional rendering of the object for the user. Digital back-propagation techniques are applied to reduce blurring in the holographic image and in the depth information, e.g., caused by modal dispersion and mode mixing in the multimode optical fiber. Some embodiments may also provide the capability for polarization-sensitive holographic imaging in different spectral regions of light.

An example embodiment of the disclosed optical imaging system may beneficially be used as a holographic endoscope for medical or industrial applications.

According to an example embodiment, provided is an apparatus, comprising: an optical router to route source light; a multimode optical fiber to transmit to the optical router image light received from a region near a remote fiber end in response to the region being illuminated with a first portion of the source light; a two-dimensional pixelated light detector; and a digital processor configured to receive light-intensity measurements made using pixels of the two-dimensional pixelated light detector; wherein the optical router is configured to cause mixing of the image light and a second portion of the source light along the two-dimensional pixelated light detector; and wherein the digital processor is configured to form a digital image with reduced speckle contrast therein by summing two or more digital images of the region, in a pixel-by-pixel manner, for different illuminations of the region.

According to another example embodiment, provided is an apparatus, comprising: an optical router to route source light; a multimode optical fiber to transmit to the optical router image light received from a region near a remote fiber end in response to the region being illuminated with a first portion of the source light; a two-dimensional pixelated light detector; and a digital processor configured to receive light-intensity measurements made using pixels of the two-dimensional pixelated light detector; wherein the optical router is configured to: (i) direct the first portion of the source light through the multimode optical fiber; (ii) make controllable changes to modal composition of the first portion of the source light to laterally move a corresponding illumination spot across the region; and (iii) cause mixing of the image light and a second portion of the source light along the two-dimensional pixelated light detector; and wherein the digital processor is configured to form a digital image using a plurality of digital images of the region corresponding to a plurality of different lateral positions of the illumination spot.

According to yet another example embodiment, provided is an apparatus, comprising: an optical router to route source light; a multimode optical fiber to transmit to the optical router image light received from a region near a remote fiber end in response to the region being illuminated with a first portion of the source light; a two-dimensional pixelated light detector; and a digital processor configured to receive time-resolved light-intensity measurements made using pixels of the two-dimensional pixelated light detector while a wavelength of the source light is being swept; wherein the optical router is configured to cause mixing of the image light and a second portion of the source light along the two-dimensional pixelated light detector; and wherein the digital processor is configured to produce data for depth-sensitive images of the region from measurements of beat frequencies obtained from the time-resolved light-intensity measurements, the beat frequencies being generated by the mixing.

### BRIEF DESCRIPTION OF THE DRAWINGS

Other aspects, features, and benefits of various disclosed embodiments will become more fully apparent, by way of example, from the following detailed description and the accompanying drawings, in which:

FIG. 1 shows a block diagram of an optical imaging system according to an embodiment;

FIG. 2 shows a transverse cross-sectional view of a multi-core optical fiber that can be used in the optical imaging system of FIG. 1 according to an embodiment;

FIG. 3 shows a front view of a pixelated photodetector that can be used in the optical imaging system of FIG. 1 according to an embodiment;

FIG. 4 shows a block diagram of an optical imaging system according to another embodiment;

FIG. 5 shows a flowchart of an acquisition method that can be used to operate the optical imaging system of FIG. 1 according to an embodiment; and

FIG. 6 shows a flowchart of an image processing method that can be used in the optical imaging system of FIG. 1 according to an embodiment.

### DETAILED DESCRIPTION

At least some embodiments disclosed herein may benefit from the use of at least some features and/or techniques disclosed in U.S. Patent Application Publication No. 2020/0200646, which is incorporated herein by reference in its entirety.

When an object is imaged through a multimode fiber, light from the object typically propagates through the fiber on different modes thereof. Due to modal dispersion and mode mixing, such a multimode optical fiber may cause the image produced by the light received from the fiber end to appear blurred.

Light propagation in a multimode fiber with mode mixing can mathematically be represented by a channel matrix H that describes the amplitude and phase relationship between the light being input to various modes at one end of the multimode fiber and the light being output from the various modes at the other end of the multimode fiber. More specifically, each matrix element Hᵢⱼ of the channel matrix H describes the amplitude and phase relationship between the j-th spatial mode at the first (e.g., proximal) end of the fiber and the light received from the i-th spatial mode at the second (e.g., distal) end of the fiber. The transposed channel matrix, i.e., H^{T}, similarly describes the amplitude and phase relationship between the light applied to the various spatial modes at the second end of the fiber and the light received from the various spatial modes at the first end of the fiber. The channel matrix H can typically be an N×N matrix, where N is the number of guided modes in the fiber.

The channel matrix H is typically a function of wavelength of light, i.e., H=H(λ). The channel matrix H may also be polarization dependent, in which case a set of two or more channel matrices H may be used to characterize light coupling between different spatial and polarization modes of the multimode fiber. Alternatively, spatial modes corresponding to different polarizations may be treated as independent modes, in which case a single channel matrix H may be used as already indicated above.

Some image-processing techniques are capable of significantly improving the quality of (e.g., removing the blur from) images obtained using light transmitted through a multimode optical fiber. Some of such image-processing techniques are referred to as back-propagation techniques. Some of such image-processing techniques rely on the knowledge of the channel matrix H of the multimode fiber through which the image is acquired.

The use of lasers in imaging systems has many benefits that may be difficult or impossible to obtain with non-laser light sources. For example, holographic imaging relies on coherent light sources (e.g., lasers) and is not practically achievable with non-coherent light sources.

One significant obstacle to laser imaging is the speckle phenomenon. Speckle arises when coherent light scattered from a surface, such as an object or a screen, is detected using a light detector. For example, if light scattered/reflected from a part of an object interferes primarily destructively at the light detector, then that part may appear as a relatively dark spot in the image. On the other hand, if light scattered from a part of an object interferes primarily constructively at the light detector, then that part may appear as a relatively bright spot in the detected image. This apparent spot-to-spot intensity variation detected even when the object or screen is uniformly lit is referred to as speckle or a speckle pattern. Since speckle superimposes a granular structure on the perceived image, which both degrades the image sharpness and annoys the viewer, speckle reduction is highly desirable.

In some embodiments, speckle reduction may be based on summing and/or averaging images having two or more independent speckle patterns. Independent speckle patterns may be produced, e.g., using diversification of phase, propagation or illumination angle(s), polarization, and/or wavelength of the illuminating laser beam. For example, wavelength diversity may reduce speckle contrast because a speckle pattern is an interference pattern whose geometric form depends on the wavelength of the illuminating light. If two wavelengths that differ by an amount indistinguishable to the human eye are used to produce the same image, then the image has a superposition of two independent speckle patterns, and the overall speckle contrast is typically reduced. Because phase, angle, polarization, and wavelength diversities are independent of one another, these techniques may be combined and used simultaneously and/or complementarily for speckle averaging and reduction.

Herein, a multimode optical fiber is able to propagate a plurality of relatively orthogonal guided modes with different lateral (transverse) intensity and/or phase profiles at the operating wavelength(s) thereof. In one example embodiment, a multimode optical fiber may have two or more optical cores in the optical cladding thereof. In another example embodiment, a multimode optical fiber may have a single optical core designed and configured to cause the normalized frequency parameter V (also referred to as the V number) associated therewith to be greater than about 2.405. In the approximation of weak guidance for generally cylindrical optical fibers, the relatively orthogonal guided modes of the fiber are conventionally referred to as the linearly polarized (LP) modes. Representative intensity and electric-field distributions of several low-order LP modes are graphically shown, e.g., in U.S. Pat. No. 8,705,913, which is incorporated herein by reference in its entirety.

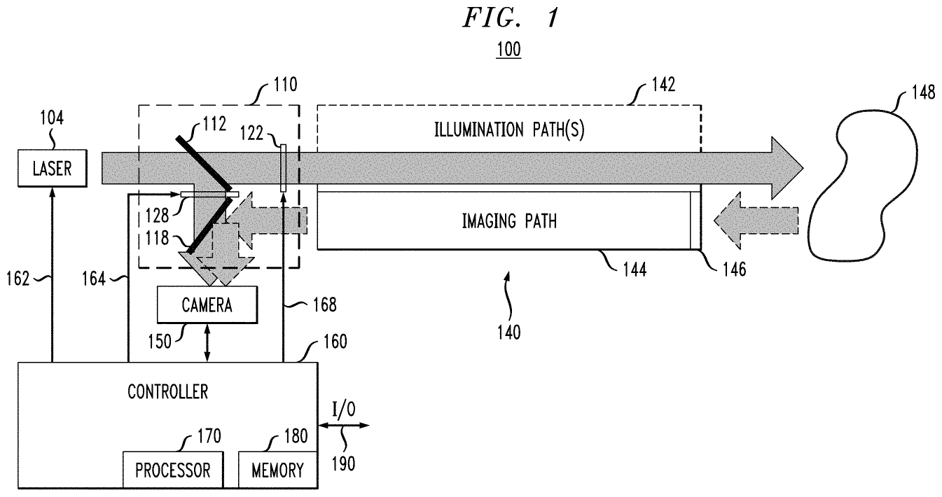

FIG. 1 shows a block diagram of an optical imaging system 100 according to an embodiment. Some embodiments of system 100 may be adapted for holographic imaging, e.g., for the purpose of remote optical imaging and characterization of objects. Some embodiments of system 100 may be operated to produce images or aid in the generation of certain images by scanning the surface of an object with a focused illumination beam, with the focusing and scanning being performed by selecting and changing the modal composition of the illumination light in a multimode optical fiber or a multi-core optical fiber. Some embodiments of system 100 may additionally be capable of functioning in an optical-reflectometer mode of operation. Example applications of system 100 may be in biomedical (e.g., endoscopic) imaging, optical component characterization, remote optical sensing, etc. In the corresponding embodiments, system 100 may be a subsystem of the larger system designed for an intended one of these specific applications or other suitable applications.

System 100 comprises a laser 104, an optical beam router 110, imaging optics 140, and a digital camera 150. An electronic controller 160 comprising a digital signal processor (DSP) 170, a memory 180, and appropriate logic and control circuitry (not explicitly shown in FIG. 1) can be used to control and/or communicate with the various components of system 100, e.g., as further described below. Raw digital images and/or reflectometer and/or optical backscattering data (e.g., in the form of image frames) captured by camera 150 can be processed using DSP 170. Memory 180 is operatively connected to DSP 170 and is configured to store therein program code(s) and raw and processed image data. System 100 further comprises an input/output (I/O) interface 190 that can be used to communicate with external circuits and/or devices. For example, I/O interface 190 may be used to export processed-image data to an external display system for rendering thereon and being viewed by the user.

In some embodiments, the output wavelength of laser 104 may be tunable via a control signal 162. In a reflectometer or optical backscattering mode of operation, laser 104 may be configured to generate controllably chirped optical pulses, in each of which the carrier frequency can be, e.g., an approximately linear function of time. In alternative embodiments, other suitable frequency-chirp functions may similarly be employed to control the generation of output light in laser 104.

In an example embodiment, optical beam router 110 may comprise a beam splitter 112, a beam combiner 118, and optical filters 122 and 128. In some embodiments, one or both of optical filters 122 and 128 may be tunable/reconfigurable, e.g., via control signals 164 and 168 applied thereto by controller 160 as indicated in FIG. 1. In some embodiments, each of optical filters 122 and 128 may be used to control and/or controllably change one or more of the following: (i) polarization of light passing therethrough; (ii) transverse intensity distribution of (i.e., the light intensity profile across) the light beam passing therethrough; and (iii) the phase profile across the light beam passing therethrough. In embodiments in which the imaging optics 140 comprises one or more multimode optical fibers, optical filters 122 and 128 may be used as mode-selective filters or mode multiplexers and/or mode demultiplexers for the illumination light and reference light, respectively.

Example optical circuits and devices that can be used to implement optical filters 122 and 128 in some embodiments are disclosed, e.g., in U.S. Pat. Nos. 8,355,638, 8,320,769, 7,174,067, and 7,639,909, and U.S. Patent Application Publication Nos. 2016/0233959 and 2015/0309249, all of which are incorporated herein by reference in their entirety. Some embodiments of optical filters 122 and 128 can benefit from the use of some optical circuits and devices disclosed in: (i) Daniele Melati, Andrea Alippi, and Andrea Melloni, “Reconfigurable Photonic Integrated Mode (De)Multiplexer for SDM Fiber Transmission,” Optics Express, 2016, v. 24, pp. 12625-12634; and (ii) Joel Carpenter and Timothy D. Wilkinson, “Characterization of Multimode Fiber by Selective Mode Excitation,” JOURNAL OF LIGHTWAVE TECHNOLOGY, vol. 30, No. 10, pp. 1386-1392, both of which are also incorporated herein by reference in their entirety.

In some embodiments, at least one of optical filters 122 and 128 can be implemented using a liquid-crystal (e.g., liquid-crystal-on-silicon, LCoS) micro-display. In such embodiments, the liquid-crystal micro-display may be operated in transmission or reflection. In some cases, different portions of the same larger liquid-crystal display may be used to implement optical filters 122 and 128, respectively.

In some embodiments, optical filters 122 and 128 can be implemented using at least some mode-selective devices that are commercially available, e.g., from CAILabs, Phoenix Photonics, and/or Kylia, as evidenced by the corresponding product-specification sheets, which are also incorporated herein by reference in their entirety.

In operation, optical beam router 110 directs illumination light from laser 104, through one or more illumination paths 142 of the imaging optics 140, to an object 148 that is being imaged. The image light backscattered and/or reflected from the object 148 is collected from the field of view of a distal end 146 of an imaging path 144 of the imaging optics 140 and delivered via the imaging path and optical beam router 110 to camera 150. Herein, the term “field of view” refers to the range of angular directions in which object 148 can be observed using camera 150 for a fixed orientation of the fiber section adjacent to the distal end 146.

Optical beam router 110 also directs reference light toward camera 150, wherein the reference light and the image light received via imaging path 144 create an interference pattern on the pixelated light detector of the camera (e.g., 300, FIG. 3). The illumination path(s) 142 and imaging path 144 may be the same or different physical optical paths. Optical (e.g., 3-dB power) splitter 112 is used to split source light applied thereto by laser 104 into two portions. The first of these two portions provides illumination light, and the second of these two portions provides reference light, which are then used as mentioned above.

In various embodiments, the imaging optics 140 may be constructed using one or more of the following optical elements: (i) one or more conventional lenses, e.g., an objective, an eyepiece, a field lens, a relay lens, etc.; (ii) an optical fiber relay; (iii) a graded-index (GRIN) rod or waveguide; and (iv) an optical fiber. In some embodiments, parts of the imaging optics 140 may be flexible, e.g., to enable insertion thereof into a bodily cavity or a difficult-to-access portion of a device under test (DUT). In some embodiments, the optical paths 142 and 144 of the imaging optics 140 may be implemented using one or more common light conduits, e.g., the same core of a multimode optical fiber. In such embodiments, a directional light coupler (not explicitly shown in FIG. 1) may be used in optical beam router 110 to appropriately spatially overlap and/or separate the illumination light and image light.

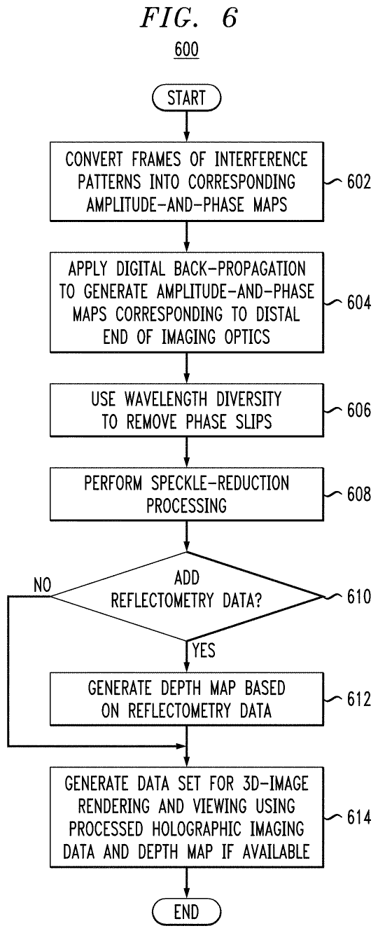

Camera 150 is configured to capture interference patterns created on the pixelated light detector thereof (e.g., 300, FIG. 3) by interference of the reference and image light beams. The resulting interference patterns can be captured in one or more image frames and the corresponding data can be stored in memory 180 and processed using DSP 170, e.g., as described in reference to FIG. 6.

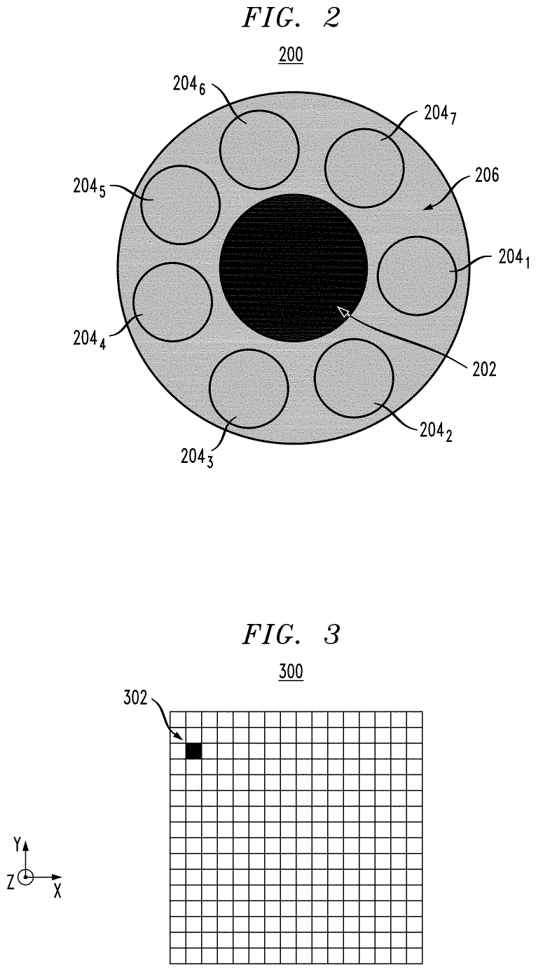

FIG. 2 shows a transverse cross-sectional view of a multi-core optical fiber 200 that can be used in imaging optics 140 (FIG. 1) according to an embodiment. As shown, optical fiber 200 comprises eight optical cores 202, 204₁-204₇ arranged in a “revolver” pattern and surrounded by an optical cladding 206. In alternative embodiments, other optical-core configurations are also possible. In the illustrated embodiment, optical core 202 has a larger diameter than the optical cores 204₁-204₇ and is capable of supporting multiple (e.g., LP) guided modes. In various embodiments, each of the optical cores 204₁-204₇ may be able to support multiple guided modes or a single guided mode and is typically constructed to have a relatively large numerical aperture (NA), e.g., NA>0.3, for better illumination of object 148 (also see FIG. 1). In operation, the optical core 202 is typically configured to provide the imaging path 144, whereas one or more of the optical cores 204₁-204₇ can typically be configured to provide the illumination path(s) 142 as indicated in FIG. 1. Different subsets of the optical cores 204₁-204₇ may be used to create different object-illumination configurations, e.g., for speckle reduction purposes and/or illumination-beam focusing and scanning.

In some embodiments, the optical cores 204₁-204₇ may be absent. In some of such embodiments, the optical core 202 may be used to provide both of the paths 142 and 144, e.g., as indicated above. For example, higher-order guided modes corresponding to the optical core 202 may be used for illumination purposes, i.e., as illumination path(s) 142, while lower-order guided modes corresponding to the optical core 202 may be used for image light, i.e., as imaging path 144.

In some embodiments, optical filter 122 can be used to dynamically adjust the spatial-mode content of the light guided by the optical core 202 and applied by the distal end 146 of the corresponding multimode optical fiber to object 148. Such adjustment of the spatial-mode content can be performed using an appropriately generated control signal 168, e.g., to adjust the focal depth of the illumination beam at object 148 and/or to laterally sweep the illumination spot across the surface of object 148. Due to the interference at object 148 of the mutually coherent light from different modes of the multimode optical fiber, certain changes of the spatial-mode content may produce the corresponding change in the size, shape, and/or position of the illumination spot on the surface of object 148. For example, the angular size of the illumination spot on the surface of object 148 may be controlled to be significantly smaller (e.g., by a factor of 10 or 100) than the field of view at the distal end 146. A tight illumination spot may be controllably moved across the surface of object 148, e.g., in a manner similar to that used in scanning microscopes, to sequentially illuminate different portions of the surface. For example, a raster scan can be implemented, wherein the illumination spot is scanned along a straight line within the field of view at the distal end 146 and is then shifted and scanned again along a parallel line.

In some embodiments, a separate light conduit, e.g., one or more additional optical fibers, may be used to provide illumination path(s) 142.

FIG. 3 shows a front view of a pixelated photodetector 300 that can be used in digital camera 150 (FIG. 1) according to an embodiment. In an example embodiment, pixelated photodetector 300 may comprise several thousand individual physical pixels, one of which is labeled in FIG. 3 using the reference numeral 302. Different ones of such pixels are typically nominally (i.e., to within fabrication tolerances) identical. In an example embodiment, each individual physical pixel 302 comprises a respective light-sensing element, e.g., a photodiode. In various embodiments, pixelated photodetector 300 can be implemented as known in the pertinent art, e.g., using a charge coupled device (CCD) or a complementary metal-oxide-semiconductor (CMOS) light sensor.

When pixelated photodetector 300 is used in system 100 for capturing holographic images, optical beam router 110 may be configured to direct the reference-light beam at a small tilt angle, i.e., not strictly orthogonally with respect to the detector's front face. In FIG. 3, the detector-face normal is parallel to the Z-coordinate axis of the shown XYZ coordinate triad. The beam tilt angle can be such, for example, that, for a planar wavefront of the reference beam, the reference-light phase linearly changes along the X-coordinate direction and is substantially constant along the Y-coordinate direction. The value of the pixel-to-pixel phase change of the reference light depends on the tilt angle and carrier wavelength and can be selected and/or measured, e.g., using a suitable calibration procedure.

In some modes of operation, two or more physical pixels 302 may be grouped to form a corresponding logical pixel, wherein the constituent physical pixels are configured to measure different relative-phase combinations of image light and reference light, which is possible due to the above-described reference-light-beam tilt angle. Such measurements can then be used, e.g., in accordance with the principles of coherent light detection, to determine both the phase and amplitude of the image light corresponding to the logical pixel. Measurements performed by different logical pixels of photodetector 300 can be used to obtain spatially resolved measurements of the phase and amplitude along the wavefront of the image light. Optical filter 128 can be used to change the polarization of the reference light, thereby enabling polarization-resolved measurements of the phase and amplitude of the image light.

For example, each logical pixel of photodetector 300 can be used to measure the following four components of the image light: (i) the in-phase component of the X-polarization, I_{X}; (ii) the quadrature component of the X-polarization, Q_{X}; (iii) the in-phase component of the Y-polarization, I_{Y}; and (iv) the quadrature component of the Y-polarization, Q_{Y}. Measurements performed using different logical pixels of photodetector 300 then provide spatially resolved measurements of these four components of the image light, e.g., I_{X}(x,y), Q_{X}(x,y), I_{Y}(x,y), and Q_{Y}(x,y), where x and y are the values of the X and Y coordinates corresponding to different logical pixels of the photodetector. As such, photodetector 300 can be operated to obtain spatially resolved measurements of the electric field vector E(x, y) of the image light, for example, based on the following formula:

$$E ( x , y ) = [ \begin{matrix}{I}_{X} ( x , y ) + j \cdot {Q}_{X} ( x , y ) \\ {I}_{Y} ( x , y ) + j \cdot {Q}_{Y} ( x , y )\end{matrix} ] \qquad ( 1 )$$

In some other embodiments, each logical pixel of photodetector 300 can be configured to measure four other linearly independent components of the image light from which the components I_{X}(x,y), Q_{X}(x,y), I_{Y}(x,y), and Q_{Y}(x,y) can be determined as appropriate linear combinations of such other measured components.

In some embodiments, each logical pixel of photodetector 300 can be used to measure the average phase and average amplitude of light received at said logical pixel at a sequence of sample times.

In the optical-reflectometer mode of operation, individual physical pixels 302 or logical pixels can be used to capture depth-imaging information, e.g., in the form of beat-frequency maps of object 148. A beat frequency can be generated by interference between the image and reference light when the carrier frequency of the output light generated by laser 104 is swept, e.g., linearly in time. Because the image and reference light have different relative times of flight to photodetector 300, the frequency sweep causes the light interference on the detector face to occur between different wavelengths of light, which causes a corresponding difference (beat) frequency to be generated in the electrical output(s) of the photodetector. The beat-frequency typically varies across the image of object 148 formed on the face of photodetector 300 due to depth variations across object 148. The corresponding beat-frequency map captures such depth variations across the image of object 148 and can be converted back into object-depth information in a relatively straightforward manner. As such, measurements performed by different logical or physical pixels of photodetector 300 can be used to obtain a depth profile of object 148, e.g., by applying a Fourier transform to the beat-frequency map. In this manner, various embodiments can provide optical-coherence-tomography image data without a need to scan the illumination light beam laterally across object 148, i.e., pixelated photodetector 300 can capture laterally wide images without such scanning of object 148.

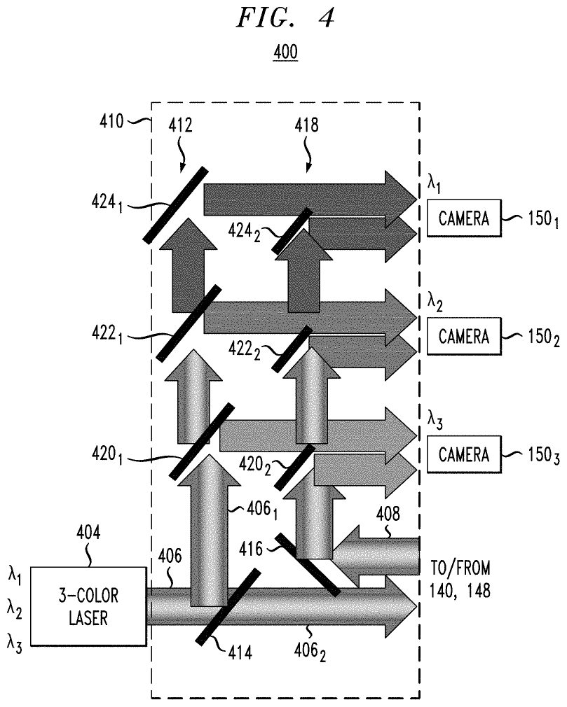

FIG. 4 shows a block diagram of an optical imaging system 400 according to another embodiment. For simplicity, imaging optics 140 and object 148 are not explicitly shown in FIG. 4.

System 400 is generally analogous to system 100 (FIG. 1), except that system 400 is capable of simultaneously capturing images of object 148 in three different wavelengths of light, λ₁, λ₂, and λ₃. Accordingly, system 400 comprises a laser source 404 capable of concurrently generating light of three different carrier wavelengths. System 400 further comprises an optical beam router 410 capable of appropriately routing illumination, image, and reference light beams of the wavelengths λ₁, λ₂, and λ₃, e.g., as indicated in FIG. 4. Digital cameras 150₁, 150₂, and 150₃ are used to separately capture the interference patterns in the wavelengths λ₁, λ₂, and λ₃, respectively. In an example embodiment, system 400 may include several appropriately positioned optical filters (not explicitly shown in FIG. 4), e.g., optical filters selective for individual ones of the wavelengths λ₁, λ₂, and λ₃, which may be functionally similar to optical filters 122 and 128 (FIG. 1).

Optical beam router 410 comprises a beam splitter 414, a turning mirror 416, and wavelength demultiplexers 412 and 418. In an example embodiment, beam splitter 414 can be a 3-dB power splitter configured to optically split an output light beam 406 generated by laser source 404 into two directionally separated sub-beams, which are labeled in FIG. 4 using the reference numerals 406₁ and 406₂. Sub-beam 406₁ is directed to wavelength demultiplexer 412. Sub-beam 406₂ is directed to imaging optics 140 and is coupled therein into one or more illumination paths 142 (also see FIG. 1). Object 148 reflects and/or backscatters sub-beam 406₂ thereby producing an image light beam 408, which is coupled into imaging path 144 of imaging optics 140 and delivered to turning mirror 416. Turning mirror 416 then redirects light beam 408 to wavelength demultiplexer 418.

As shown in FIG. 4, wavelength demultiplexer 412 comprises wavelength-selective beam splitters 420₁ and 422₄ and a mirror 424₁. Wavelength-selective beam splitter 420₁ is configured to receive sub-beam 406₁ and operates to direct light of wavelength λ₃ to camera 150₃ and to direct light of wavelengths λ₁ and λ₂ to wavelength-selective beam splitter 422₁. Wavelength-selective beam splitter 422₁ operates to direct light of wavelength λ₂ to camera 150₂ and to direct light of wavelength λ₁ to mirror 424₁. Mirror 424₁ operates to direct the light of wavelength λ₁ received from wavelength-selective beam splitter 422₁ to camera 150₁. The orientation of beam splitters 420₁ and 422₁ and mirror 424₁ may be such that each of the corresponding light beams impinges onto the front face of the corresponding pixelated detector 300 at a small tilt angle, e.g., as explained above in reference to FIG. 3.

As shown in FIG. 4, wavelength demultiplexer 418 is similar to wavelength demultiplexer 412 and comprises wavelength-selective beam splitters 420₂ and 422₂ and a mirror 424₂. Wavelength-selective beam splitter 420₂ is configured to receive beam 408 from turning mirror 416 and operates to direct light of wavelength λ₃ to camera 150₃ and to direct light of wavelengths λ₁ and λ₂ to wavelength-selective beam splitter 422₂. Wavelength-selective beam splitter 422₂ operates to direct light of wavelength λ₂ to camera 150₂ and to direct light of wavelength λ₁ to mirror 424₂. Mirror 424₂ operates to direct the light of wavelength λ₁ received from wavelength-selective beam splitter 422₂ to camera 150₁. The orientation of beam splitters 420₂ and 422₂ and mirror 424₂ may be such that each of the corresponding light beams impinges onto the front face of the corresponding pixelated detector 300 approximately orthogonally (e.g., along the surface normal).

In alternative embodiments, other suitable designs of wavelength demultiplexers 412 and 418 may also be used.

Each of cameras 150₁, 150₂, and 150₃ is configured to capture interference patterns created on the pixelated light detector thereof (e.g., 300, FIG. 3) by interference of the corresponding reference and image light beams, thereby capturing interference patterns corresponding to wavelengths λ₁, λ₂, and λ₃, respectively. The captured interference patterns may be stored in memory 180 and processed using DSP 170, e.g., as described below.

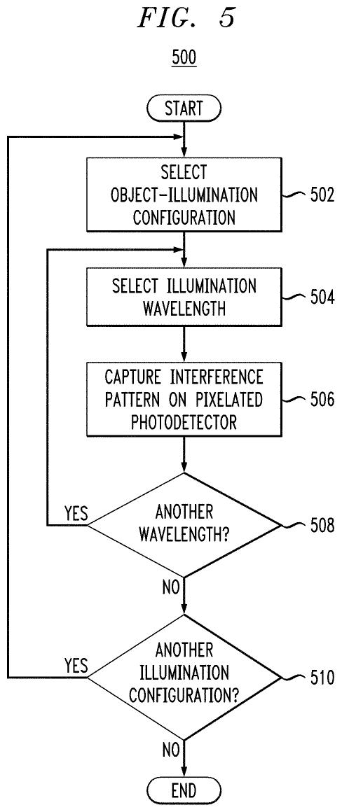

FIG. 5 shows a flowchart of an acquisition method 500 according to an embodiment. Method 500 can be used, e.g., to operate system 100 (FIG. 1) in a holographic-imaging mode. Based on the provided description, a person of ordinary skill in the art will be able to adapt method 500 to operating system 400 (FIG. 4) without any undue experimentation.

At step 502, optical beam router 110 is configured to select a light-routing configuration for directing an illumination light beam from laser 104, through one or more illumination paths 142, to object 148. For example, in some embodiments, the selected illumination path(s) 142 may include a selected subset of optical cores 204₁-204₇ of fiber 200 (FIG. 2). In some other embodiments, the selected illumination path 142 may include one or more selected higher-order transverse guided modes corresponding to the optical core 202.

In some embodiments, different instances of step 502 may be used to change the position of a tight illumination spot on the surface of object 148, e.g., to perform a raster scan thereof.

At step 504, controller 160 generates an appropriate control signal 162 to cause laser 104 to generate an illumination light beam having a selected wavelength λ and direct the generated light beam to optical beam router 110, wherein the illumination light is routed using the light-routing configuration selected at step 502.

At step 506, controller 160 operates camera 150 to capture one or more image frames to record the interference pattern created, e.g., as explained above, on the pixelated photodetector 300 of the camera. The captured image frame(s) may then be stored in memory 180 for further processing, e.g., as described in reference to FIG. 6.

Step 508 controls wavelength changes that might be needed for speckle reduction. If the wavelength λ selected at the previous instance of step 504 needs to be changed, then the processing of method 500 is directed back to step 504. Otherwise, the processing of method 500 is directed to step 510.

Step 510 controls illumination-configuration changes that might be needed for speckle reduction and/or illumination-beam focusing and scanning.

For example, speckle reduction may involve varying the illumination light beam(s), in time, and then superimposing captured images to reduce speckle patterning by the resulting time averaging. Such a time-dependent variations of the illumination light bean may include varying the wavelength(s) of the illumination light, varying the illumination of the optical cores 204₁-204₇ (see FIG. 2) and/or the core selection thereof, varying the optical mode content of the illumination light beam carried by a multimode optical fiber and/or varying the polarization content of the illumination light beam, e.g., using optical filter 122.

If the illumination-configuration selected at the previous instance of step 502 needs to be changed, e.g., to provide time variation and/or scanning of the illumination beam, then the processing of method 500 is directed from step 510 back to step 502. Otherwise, the processing of method 500 is terminated.

FIG. 6 shows a flowchart of an image processing method 600 that can be used in system 100 according to an embodiment. Method 600 represents example image processing corresponding to the same scene or object 148.

At step 602, DSP 170 converts each captured 2-dimensional interference-pattern frame into the corresponding amplitude-and-phase map, e.g., for one or two relatively orthogonal polarizations. In an example embodiment, the conversion can be performed, e.g., as explained above in reference to Eq. (1). For a fixed polarization, the contents E(x, y) of each of such amplitude-and-phase maps Mₙ(λₙ, Λₙ) can be expressed, for example, using the following formula:

E(x,y)=A(x,y)·exp(j·φ(x,y)) (2)

where n=1, 2, . . . , N; N is the total number of captured frames for the scene or object 148; λₙ is the illumination wavelength corresponding to the n-th frame; Λₙ is the illumination configuration corresponding to the n-th frame; A is the real-valued amplitude; φ is the phase (0≤φ<2π), and (x,y) are the coordinates of the corresponding physical or logical pixel of photodetector 300.

For step 602, separate sets of frames may, in some embodiments, be captured for the two orthogonal polarization directions, e.g., the relatively orthogonal directions X and Y along the 2-dimensional pixelated array of the photodetector 300. Such separate frames may be captured, e.g., by using step 502 of method 500 to relatively rotate the polarization of the reference light beam, e.g., by about 90 degrees, for the images of different polarization. Such embodiments may be used to produce polarization-sensitive images and/or may be used to recover phases and amplitudes of individual guided modes at the photodetector 300, e.g., as discussed below.

At step 604, DSP 170 applies a suitable back-propagation algorithm to each of the maps Mₙ to generate the corresponding corrected maps M′ₙ(λₙ, Λₙ). In an example embodiment, the back-propagation algorithm may be based on the above-mentioned channel matrix H of the imaging optics 140. As already indicated above, the channel matrix H can be measured using a suitable calibration method. In other embodiments, other suitable back-propagation algorithms known to persons of ordinary skill in the pertinent art may also be used in step 604 for the conversion of the map Mₙ into the corresponding corrected map M′ₙ.

In some embodiments, such back-propagation may be performed based on the measured content of propagation modes at the pixelated array of the photodetector 300. That is, the measured phase and amplitude map of a captured frame may be used to reconstruct the complex superposition of propagating modes at the pixelated array of the photodetector 300, e.g., for a complete orthonormal basis of such modes. Determining such a superposition typically involves determining phases and amplitudes of the contributions of said individual modes to the measured light pattern at the pixelated array of the photodetector 300, e.g., by numerically evaluating overlap integrals for the various modes with said measured complex light pattern. Then, the complex superposition of propagating modes can be back-propagated with a pre-determined channel matrix for the imaging path 144 to obtain the complex superposition of propagating modes over a lateral surface at the remote end of the imaging path 144, i.e., near object 148. Such back-propagation can remove, e.g., image defects caused by different propagation characteristics of various modes in the imaging path 144, e.g., different velocities and/or attenuation, and caused by mode mixing in the imaging path 144, e.g., due to fiber bends.

The contents E′(x′,y′) of each of such corrected maps M′ₙ(λₙ, Λₙ) can be expressed, for example, using the following formula:

E′(x′,y′)=A′(x′,y′)·exp(j·φ′(x′,y′)) (3)

where A′ is the corrected amplitude; φ′ is the corrected phase (0≤φ′<2π), and (x′,y′) are the coordinates in the image-input plane at the distal end of the imaging optics 140, i.e., the end proximal to the scene or object 148. Due to the fringe effects, the ranges for the coordinates x′ and y′ may be narrower than the ranges for the coordinates x and y. In Eq. (3), polarization dependence is not explicitly shown, but a person of ordinary skill in the pertinent art would understand how such polarization dependence can be included, e.g., by A′ having separate components for two orthogonal polarizations and possibly φ′ being polarization dependent.

At step 606, the corrected maps M′ₙ(λₙ, Λₙ) corresponding to different wavelengths λₙ, but to the same polarization Pₙ and the same illumination configuration Λₙ may be cross-checked for consistency and, if warranted, the corrected maps M′ₙ may be converted into the corresponding corrected maps M″ₙ. The contents \tilde{E}(x′,y′) of each of such corrected maps M″ₙ(λₙ, Pₙ, Λₙ) can be expressed, for example, using the following formula:

\tilde{E}(x′,y′)=A′(x′,y′)·exp(j·Φ(x′,y′)) (4)

where Φ is the “absolute” phase, the values of which are no longer limited to the interval [0,2π). A person of ordinary skill in the art will understand that the processing implemented at step 606 may be directed at eliminating the so-called phase slips. Phase slips can be eliminated, e.g., by comparing the phase data corresponding to wavelengths sufficiently different from one another, because the phase slips, if present, typically occur at different locations for such different wavelengths.

In some embodiments, step 606 may be optional (e.g., not present).

At step 608, DSP 170 performs speckle-reduction processing. In an example embodiment, some groups of the corrected 2-dimensional maps M″ₙ(λₙ, Λₙ) may be fused together or superimposed, e.g., by summation, to generate corresponding fused maps in a (logical pixel)-by-(logical pixel) manner. A group of maps M″ₙ(λₙ, Λₙ) suitable for such summation typically has maps corresponding to the image frames captured for a specific purpose of speckle reduction, e.g., as a result of time variations of the illumination light beam. The conditions under which those image frames may be acquired are typically characterized by (i) relatively small differences of the respective illumination wavelengths λₙ and (ii) different respective illumination configurations Λₙ, e.g., lateral propagation-mode composition and/or polarization, as already discussed. As already explained above, summing and/or averaging the images corresponding to two or more independent speckle configurations typically results in a significant reduction of speckle contrast.

Note that some corrected maps M″ₙ are neither suitable nor intended for summation. For example, the corrected maps M″ₙ corresponding to the wavelengths that differ by a relatively large Δλ are not intended for speckle-reduction purposes. Rather, such frames are typically acquired to capture some wavelength-dependent characteristics (e.g., different colors) of the imaged scene or object 148.

Step 610 is used to determine whether or not optical-reflectometry data are to be included in the final output. For example, if optical-reflectometry data were acquired for the corresponding scene or object 148, then the processing of method 600 may be directed to step 612. Otherwise, the processing of method 600 may be directed to step 614.

In some embodiments, steps 610 and 612 may be optional (e.g., not present).

At step 612, the optical-reflectometry data are converted into a depth map z(x′,y′). Herein, z is the relative “height” of the part of object or scene 148 having the coordinates (x′,y′) with respect to a reference plane. In an example embodiment, such reference plane may be the image-input plane at the distal end of the imaging optics 140 and may be about perpendicular to the light propagation direction in the proximate section of imaging path 144. As already mentioned above, a depth map z(x′,y′) can be obtained by applying a Fourier transform to a corresponding beat-frequency map acquired in an optical-reflectometer mode of operation of system 100.

At step 614, the processed holographic-imaging data and, if available, processed optical-reflectometry data are combined into a data file suitable for convenient image rendering and viewing. In an example embodiment, the data file generated at step 614 enables the user to view a 3-dimensional (e.g., resolved in x, y, and z spatial dimensions) image of the corresponding scene or object 148 with at least some characteristics of the image scene or object being also resolved in polarization and/or wavelength.

According to an example embodiment disclosed above, e.g., in the summary section and/or in reference to any one or any combination of some or all of FIGS. 1-6, provided is an apparatus comprising: an optical router (e.g., 110, FIG. 1; 410, FIG. 4) to route source light; a multimode optical fiber (e.g., 200, FIG. 2) to transmit to the optical router image light received from a region (e.g., 148, FIG. 1) near a remote fiber end in response to the region being illuminated with a first portion of the source light; a two-dimensional pixelated light detector (e.g., 150, FIG. 1; 300, FIG. 3); and a digital processor (e.g., 160/170, FIG. 1) configured to receive light-intensity measurements made using pixels of the two-dimensional pixelated light detector; wherein the optical router is configured to cause mixing of the image light and a second portion of the source light along the two-dimensional pixelated light detector; and wherein the digital processor is configured to form a digital image with reduced speckle contrast therein by summing (e.g., at 608, FIG. 6) two or more digital images of the region, in a pixel-by-pixel manner, for different illuminations of the region.

In some embodiments of the above apparatus, the apparatus is configured to make controllable changes of one or more of phase, angle, polarization, modal composition, and wavelength of the first portion of the source light to cause the two or more digital images to have different speckle patterns therein.

In some embodiments of any of the above apparatus, the apparatus further comprises a tunable laser (e.g., 104, FIG. 1) configured to generate the source light.

In some embodiments of any of the above apparatus, the tunable laser is capable of sweeping a wavelength of the source light while pixels of the two-dimensional pixelated light detector are performing time-resolved light-intensity measurements for measuring beat frequencies generated by the mixing; and wherein the digital processor is configured to produce (e.g., at 612, FIG. 6) data for depth-sensitive images of the region using the measured beat frequencies.

In some embodiments of any of the above apparatus, the digital processor is configured to apply digital back-propagation (e.g., at 604, FIG. 6) to the two or more digital images of the region.

In some embodiments of any of the above apparatus, the apparatus is configured to obtain spatially resolved measurements of amplitude and phase (e.g., A(x,y), φ(x,y), Eq. (2)) of the image light along the two-dimensional pixelated light detector.

In some embodiments of any of the above apparatus, the digital processor is configured to correct phase slips (e.g., at 606, FIG. 6) in the measurements of the phase based on digital images corresponding to different wavelengths of the source light.

In some embodiments of any of the above apparatus, the optical router comprises a polarization filter (e.g., 122, 128, FIG. 1) configured to filter at least one of the first and second portions of the source light.

In some embodiments of any of the above apparatus, the optical router is configured to direct the first portion of the source light through the multimode optical fiber.

In some embodiments of any of the above apparatus, the optical router comprises a mode-selective filter (e.g., 122, FIG. 1) configured to selectively couple the first portion of the source light into a selected set of guided modes of a proximate section of the multimode optical fiber.

In some embodiments of any of the above apparatus, the multimode optical fiber has a plurality of optical cores (e.g., 204₁-204₇, FIG. 2) for guiding the first portion of the source light to the region.

In some embodiments of any of the above apparatus, the optical router comprises a wavelength demultiplexer (e.g., 412, 418, FIG. 4) configured to spatially separate light of two or more different wavelengths (e.g., λ₁, λ₂, λ₃, FIG. 4) present in the source light.

In some embodiments of any of the above apparatus, the apparatus is configurable to perform optical reflectometry measurements of the region using the multimode optical fiber and the two-dimensional pixelated light detector.

According to another example embodiment disclosed above, e.g., in the summary section and/or in reference to any one or any combination of some or all of FIGS. 1-6, provided is an apparatus comprising: an optical router (e.g., 110, FIG. 1; 410, FIG. 4) to route source light; a multimode optical fiber (e.g., 200, FIG. 2) to transmit to the optical router image light received from a region (e.g., 148, FIG. 1) near a remote fiber end in response to the region being illuminated with a first portion of the source light; a two-dimensional pixelated light detector (e.g., 150, FIG. 1; 300, FIG. 3); and a digital processor (e.g., 160/170, FIG. 1) configured to receive light-intensity measurements made using pixels of the two-dimensional pixelated light detector; wherein the optical router is configured to: (i) direct the first portion of the source light through the multimode optical fiber; (ii) make controllable changes to modal composition of the first portion of the source light to laterally move across the region a corresponding illumination spot formed therein; and (iii) cause mixing of the image light and a second portion of the source light along the two-dimensional pixelated light detector; and wherein the digital processor is configured to form a digital image using a plurality of digital images of the region corresponding to a plurality of different lateral positions of the illumination spot.

In some embodiments of the above apparatus, the optical router comprises a mode-selective filter (e.g., 122, FIG. 1) configured to selectively couple the first portion of the source light into a selected set of guided modes of a proximate section of the multimode optical fiber.

In some embodiments of any of the above apparatus, a size of the illumination spot is smaller than a field of view at the remote fiber end.

In some embodiments of any of the above apparatus, the digital processor is configured to apply digital back-propagation (e.g., at 604, FIG. 6) to the plurality digital images of the region.

In some embodiments of any of the above apparatus, the apparatus is configured to raster-scan the illumination spot across the surface of object 148.

According to yet another example embodiment disclosed above, e.g., in the summary section and/or in reference to any one or any combination of some or all of FIGS. 1-6, provided is an apparatus comprising: an optical router (e.g., 110, FIG. 1; 410, FIG. 4) to route source light; a multimode optical fiber (e.g., 200, FIG. 2) to transmit to the optical router image light received from a region (e.g., 148, FIG. 1) near a remote fiber end in response to the region being illuminated with a first portion of the source light; a two-dimensional pixelated light detector (e.g., 150, FIG. 1; 300, FIG. 3); and a digital processor (e.g., 160/170, FIG. 1) configured to receive time-resolved light-intensity measurements made using pixels of the two-dimensional pixelated light detector while a wavelength of the source light is being swept; wherein the optical router is configured to cause mixing of the image light and a second portion of the source light along the two-dimensional pixelated light detector; and wherein the digital processor is configured to produce (e.g., at 612, FIG. 6) data for depth-sensitive images of the region from measurements of beat frequencies obtained from the time-resolved light-intensity measurements, the beat frequencies being generated by the mixing.

In some embodiments of the above apparatus, the apparatus further comprises a tunable laser (e.g., 104, FIG. 1) configured to generate the source light while sweeping the wavelength thereof.

In some embodiments of any of the above apparatus, the digital processor is configured to form a digital image with reduced speckle contrast therein by summing (e.g., at 608, FIG. 6) two or more digital images of the region, in a pixel-by-pixel manner, for different illuminations of the region.

In some embodiments of any of the above apparatus, the apparatus is configured to make controllable changes of one or more of phase, angle, polarization, modal composition, and wavelength of the first portion of the source light to cause the two or more digital images to have different speckle patterns therein.

In some embodiments of any of the above apparatus, the digital processor is configured to apply digital back-propagation (e.g., at 604, FIG. 6) to a depth map of the region to produce said data, the depth map being generated using the measurements of the beat frequencies corresponding to different pixels of the two-dimensional pixelated light detector.

In some embodiments of any of the above apparatus, the optical router is configured to direct the first portion of the source light through the multimode optical fiber.

In some embodiments of any of the above apparatus, the multimode optical fiber has a plurality of optical cores (e.g., 204₁-204₇, FIG. 2) for guiding the first portion of the source light to the region.

In some embodiments of any of the above apparatus, the apparatus is configured to perform optical reflectometry measurements of the region to obtain the measurements of the beat frequencies.

While this disclosure includes references to illustrative embodiments, this specification is not intended to be construed in a limiting sense. Various modifications of the described embodiments, as well as other embodiments within the scope of the disclosure, which are apparent to persons skilled in the art to which the disclosure pertains are deemed to lie within the principle and scope of the disclosure, e.g., as expressed in the following claims.

Unless explicitly stated otherwise, each numerical value and range should be interpreted as being approximate as if the word “about” or “approximately” preceded the value or range.

It will be further understood that various changes in the details, materials, and arrangements of the parts which have been described and illustrated in order to explain the nature of this disclosure may be made by those skilled in the art without departing from the scope of the disclosure, e.g., as expressed in the following claims.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

Reference herein to “one embodiment” or “an embodiment” means that a particular feature, structure, or characteristic described in connection with the embodiment can be included in at least one embodiment of the disclosure. The appearances of the phrase “in one embodiment” in various places in the specification are not necessarily all referring to the same embodiment, nor are separate or alternative embodiments necessarily mutually exclusive of other embodiments. The same applies to the term “implementation.”

Unless otherwise specified herein, the use of the ordinal adjectives “first,” “second,” “third,” etc., to refer to an object of a plurality of like objects merely indicates that different instances of such like objects are being referred to, and is not intended to imply that the like objects so referred-to have to be in a corresponding order or sequence, either temporally, spatially, in ranking, or in any other manner.

Unless otherwise specified herein, in addition to its plain meaning, the conjunction “if” may also or alternatively be construed to mean “when” or “upon” or “in response to determining” or “in response to detecting,” which construal may depend on the corresponding specific context. For example, the phrase “if it is determined” or “if [a stated condition] is detected” may be construed to mean “upon determining” or “in response to determining” or “upon detecting [the stated condition or event]” or “in response to detecting [the stated condition or event].”

Throughout the detailed description, the drawings, which are not to scale, are illustrative only and are used in order to explain, rather than limit the disclosure. The use of terms such as height, length, width, top, bottom, is strictly to facilitate the description of the embodiments and is not intended to limit the embodiments to a specific orientation. For example, height does not imply only a vertical rise limitation, but is used to identify one of the three dimensions of a three dimensional structure as shown in the figures. Such “height” would be vertical where the reference plane horizontal but would be horizontal where the reference plane is vertical, and so on.

Also for purposes of this description, the terms “couple,” “coupling,” “coupled,” “connect,” “connecting,” or “connected” refer to any manner known in the art or later developed in which energy is allowed to be transferred between two or more elements, and the interposition of one or more additional elements is contemplated, although not required. Conversely, the terms “directly coupled,” “directly connected,” etc., imply the absence of such additional elements. The same type of distinction applies to the use of terms “attached” and “directly attached,” as applied to a description of a physical structure. For example, a relatively thin layer of adhesive or other suitable binder can be used to implement such “direct attachment” of the two corresponding components in such physical structure.

The described embodiments are to be considered in all respects as only illustrative and not restrictive. In particular, the scope of the disclosure is indicated by the appended claims rather than by the description and figures herein. All changes that come within the meaning and range of equivalency of the claims are to be embraced within their scope.

A person of ordinary skill in the art would readily recognize that at least some steps of method 600 can be performed by programmed computers. Herein, some embodiments are intended to cover program storage devices, e.g., digital data storage media, which are machine or computer readable and encode machine-executable or computer-executable programs of instructions where said instructions perform some or all of the steps of methods described herein. The program storage devices may be, e.g., digital memories, magnetic storage media such as a magnetic disks or tapes, hard drives, or optically readable digital data storage media. The embodiments are also intended to cover computers programmed to perform said steps of methods described herein.

The description and drawings merely illustrate the principles of the disclosure. It will thus be appreciated that those of ordinary skill in the art will be able to devise various arrangements that, although not explicitly described or shown herein, embody the principles of the disclosure and are included within its scope. Furthermore, all examples recited herein are principally intended expressly to be only for pedagogical purposes to aid the reader in understanding the principles of the disclosure and the concepts contributed by the inventor(s) to furthering the art, and are to be construed as being without limitation to such specifically recited examples and conditions. Moreover, all statements herein reciting principles, aspects, and embodiments of the disclosure, as well as specific examples thereof, are intended to encompass equivalents thereof.

The functions of the various elements shown in the figures, including any functional blocks labeled as “processors” and/or “controllers,” may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), and non volatile storage. Other hardware, conventional and/or custom, may also be included. Similarly, any switches shown in the figures are conceptual only. Their function may be carried out through the operation of program logic, through dedicated logic, through the interaction of program control and dedicated logic, or even manually, the particular technique being selectable by the implementer as more specifically understood from the context.

As used in this application, the term “circuitry” may refer to one or more or all of the following: (a) hardware-only circuit implementations (such as implementations in only analog and/or digital circuitry); (b) combinations of hardware circuits and software, such as (as applicable): (i) a combination of analog and/or digital hardware circuit(s) with software/firmware and (ii) any portions of hardware processor(s) with software (including digital signal processor(s)), software, and memory(ies) that work together to cause an apparatus, such as a mobile phone or server, to perform various functions); and (c) hardware circuit(s) and or processor(s), such as a microprocessor(s) or a portion of a microprocessor(s), that requires software (e.g., firmware) for operation, but the software may not be present when it is not needed for operation.” This definition of circuitry applies to all uses of this term in this application, including in any claims. As a further example, as used in this application, the term circuitry also covers an implementation of merely a hardware circuit or processor (or multiple processors) or portion of a hardware circuit or processor and its (or their) accompanying software and/or firmware. The term circuitry also covers, for example and if applicable to the particular claim element, a baseband integrated circuit or processor integrated circuit for a mobile device or a similar integrated circuit in server, a cellular network device, or other computing or network device.

“SUMMARY OF SOME SPECIFIC EMBODIMENTS” in this specification is intended to introduce some example embodiments, with additional embodiments being described in “DETAILED DESCRIPTION” and/or in reference to one or more drawings. “SUMMARY OF SOME SPECIFIC EMBODIMENTS” is not intended to identify essential elements or features of the claimed subject matter, nor is it intended to limit the scope of the claimed subject matter.

What is claimed is:

1. An apparatus, comprising:
an optical router to route source light;
a multimode optical fiber to transmit to the optical router image light received from a region near a remote fiber end in response to the region being illuminated with a first portion of the source light;
a two-dimensional pixelated light detector; and
a digital processor configured to receive light-intensity measurements made using pixels of the two-dimensional pixelated light detector;
wherein the optical router is configured to cause mixing of the image light and a second portion of the source light along the two-dimensional pixelated light detector; and
wherein the digital processor is configured to form a digital image with reduced speckle contrast therein by summing two or more digital images of the region, in a pixel-by-pixel manner, for different illuminations of the region.

2. The apparatus of claim 1, wherein the apparatus is configured to make controllable changes of one or more of phase, angle, polarization, modal composition, and wavelength of the first portion of the source light to cause the two or more digital images to have different speckle patterns therein.

3. The apparatus of claim 1, further comprising a tunable laser configured to generate the source light.

4. The apparatus of claim 3,
wherein the tunable laser is capable of sweeping a wavelength of the source light while pixels of the two-dimensional pixelated light detector are performing time-resolved light-intensity measurements for measuring beat frequencies generated by the mixing; and
wherein the digital processor is configured to produce data for depth-sensitive images of the region using the measured beat frequencies.

5. The apparatus of claim 1, wherein the digital processor is configured to apply digital back-propagation to the two or more digital images of the region.

6. The apparatus of claim 1, wherein the apparatus is configured to obtain spatially resolved measurements of amplitude and phase of the image light along the two-dimensional pixelated light detector.

7. The apparatus of claim 6, wherein the digital processor is configured to correct phase slips in the measurements of the phase based on digital images corresponding to different wavelengths of the source light.

8. The apparatus of claim 1, wherein the optical router comprises a polarization filter configured to filter at least one of the first and second portions of the source light.

9. The apparatus of claim 1, wherein the optical router is configured to direct the first portion of the source light through the multimode optical fiber.

10. The apparatus of claim 9, wherein the optical router comprises a mode-selective filter configured to selectively couple the first portion of the source light into a selected set of guided modes of a proximate section of the multimode optical fiber.

11. The apparatus of claim 1, wherein the multimode optical fiber has a plurality of optical cores for guiding the first portion of the source light to the region.

12. The apparatus of claim 1, wherein the optical router comprises a wavelength demultiplexer configured to spatially separate light of two or more different wavelengths present in the source light.

13. The apparatus of claim 1, wherein the apparatus is configurable to perform optical reflectometry measurements of the region using the multimode optical fiber and the two-dimensional pixelated light detector.

14. An apparatus, comprising:
an optical router to route source light;
a multimode optical fiber to transmit to the optical router image light received from a region near a remote fiber end in response to the region being illuminated with a first portion of the source light;
a two-dimensional pixelated light detector; and
a digital processor configured to receive light-intensity measurements made using pixels of the two-dimensional pixelated light detector;
wherein the optical router is configured to:
direct the first portion of the source light through the multimode optical fiber;
make controllable changes to modal composition of the first portion of the source light to laterally move a corresponding illumination spot across the region; and
cause mixing of the image light and a second portion of the source light along the two-dimensional pixelated light detector; and
wherein the digital processor is configured to form a digital image using a plurality of digital images of the region corresponding to a plurality of different lateral positions of the illumination spot.

15. The apparatus of claim 14, wherein the optical router comprises a mode-selective filter configured to selectively couple the first portion of the source light into a selected set of guided modes of a proximate section of the multimode optical fiber.

16. The apparatus of claim 14, wherein a size of the illumination spot is smaller than a field of view at the remote fiber end.

17. The apparatus of claim 14, wherein the digital processor is configured to apply digital back-propagation to the plurality digital images of the region.

18. The apparatus of claim 14, wherein the apparatus is configured to raster-scan the illumination spot.

19. An apparatus, comprising:
an optical router to route source light;
a multimode optical fiber to transmit to the optical router image light received from a region near a remote fiber end in response to the region being illuminated with a first portion of the source light;
a two-dimensional pixelated light detector; and
a digital processor configured to receive time-resolved light-intensity measurements made using pixels of the two-dimensional pixelated light detector while a wavelength of the source light is being swept;
wherein the optical router is configured to cause mixing of the image light and a second portion of the source light along the two-dimensional pixelated light detector; and
wherein the digital processor is configured to produce data for depth-sensitive images of the region from measurements of beat frequencies obtained from the time-resolved light-intensity measurements, the beat frequencies being generated by the mixing.

20. The apparatus of claim 19, further comprising a tunable laser configured to generate the source light while sweeping the wavelength thereof.

21. The apparatus of claim 19, wherein the digital processor is configured to form a digital image with reduced speckle contrast therein by summing two or more digital images of the region, in a pixel-by-pixel manner, for different illuminations of the region.

22. The apparatus of claim 21, wherein the apparatus is configured to make controllable changes of one or more of phase, angle, polarization, modal composition, and wavelength of the first portion of the source light to cause the two or more digital images to have different speckle patterns therein.

23. The apparatus of claim 19, wherein the digital processor is configured to apply digital back-propagation to a depth map of the region to produce said data, the depth map being generated using the measurements of the beat frequencies corresponding to different pixels of the two-dimensional pixelated light detector.

24. The apparatus of claim 19, wherein the optical router is configured to direct the first portion of the source light through the multimode optical fiber.

25. The apparatus of claim 24, wherein the multimode optical fiber has a plurality of optical cores for guiding the first portion of the source light to the region.

26. The apparatus of claim 19, wherein the apparatus is configured to perform optical reflectometry measurements of the region to obtain the measurements of the beat frequencies.
